(19) United States
(12) Patent Application Publication
(10) Pub. No.: US 2025/0257348 A1
(43) Pub. Date: Aug. 14, 2025

(54) METHOD FOR PRODUCING LACTIC ACID BACTERIA ENTRAPPED IN CALCIUM-ALGINATE BEADS

(71) Applicant: CJ CHEILJEDANG CORPORATION, Seoul (KR)

(72) Inventors: Dongjoo SHIN, Seoul (KR); Min Kyeong SHIN, Seoul (KR); Kyeong Soo KIM, Seoul (KR); Tae Hyun KIM, Seoul (KR); Ah Jin KIM, Seoul (KR)

(73) Assignee: CJ CHEILJEDANG CORPORATION, Seoul (KR)

(21) Appl. No.: 18/856,370

(22) PCT Filed: Apr. 13, 2023
(86) PCT No.: PCT/KR2023/004987

§ 371 Date: Oct. 11, 2024

(30) Foreign Application Priority Data
Apr. 13, 2022 (KR) 10-2022-0045756

(51) Int. Cl. C12N 11/04; C12N 1/20; C12N 11/10

(52) U.S. Cl. CPC C12N 11/04; C12N 1/20; C12N 11/10

(57) ABSTRACT

The present disclosure relates to a method for producing lactic acid bacteria encapsulated with calcium-alginate beads; lactic acid bacteria encapsulated with calcium-alginate beads, which are produced by the method above; a method for improving the production yield of calcium-alginate beads for encapsulating lactic acid bacteria; and a method for improving the survival rate of freeze-dried lactic acid bacteria.

### TECHNICAL FIELD

The present disclosure relates to a method for producing lactic acid bacteria encapsulated with calcium-alginate beads; lactic acid bacteria encapsulated with calcium-alginate beads, which are produced by the method above; a method for improving the production yield of calcium-alginate beads for encapsulating lactic acid bacteria; and a method for improving the survival rate of freeze-dried lactic acid bacteria.

### BACKGROUND ART

The lactic acid bacteria are also called lactic bacteria or Lactobacillus, and live in the intestines of mammals and prevent abnormal fermentation by saprophytes. Thus, they are utilized as medicine for intestinal disorders. For example, L. bulgaricus is a lactic acid bacterium that has been known from long ago, and is used for the production of yogurt and also as a starter when producing cheese or cultured butter. Additionally, the aerobic lactic acid bacterium L. acidophilus, which is found in the intestines of all mammals including humans and other animals, is used for the production of butter or milk or for the treatment of autointoxication. Further, L. lactis produces DL-lactic acid and is used for the production of butter or cheese because it is always found in milk. L. lactis is the most important bacterial species for dairy applications.

Such beneficial lactic acid bacteria reside in the intestine and exhibit various physiological activities, such as activation of intestinal movement, inhibition of harmful bacteria, promotion of production of vitamins and immunostimulants, amelioration of atopic dermatitis, etc. However, such physiological activities are exhibited only when a much larger amount of lactic acid bacteria than that intake from foods such as yogurt, etc. is consumed. Thus, readily edible forms (e.g., powders and capsules) of lactic acid bacterial isolates are currently commercially available. However, when lactic acid bacteria are processed in the form of powders or capsules, they may not exhibit the inherent physiological activities because most of them are likely to be killed during long-term storage, which is a disadvantage.

In general, lactic acid bacteria are produced in powder form by a freeze-drying method or a spray-drying method. Among them, the freeze-drying method is a method in which microorganisms are suspended in a freeze-drying suspension solution, frozen, and then dried under reduced pressure. However, since lactic acid bacteria are anaerobic and very sensitive to the surrounding environment, the viability of powdered lactic acid bacteria is reduced at low temperature and room temperature, thereby reducing the number of viable bacteria. Recently, in order to overcome such disadvantages, recent efforts have been made to develop methods for coating lactic acid bacteria with various coating materials. For example, methods for maintaining the quality during storage using starch, gelatin, alginic acid, cellulose, hardened oil, various emulsifiers, etc., are used as cryoprotectants, etc.

Live lactic acid bacteria are killed by an increase in temperature; thus, freeze-drying is the most effective method to maintain cell activity during storage of lactic acid bacteria. Although freeze-drying can greatly improve the storage and distribution of lactic acid bacteria, the survival rate of lactic acid bacteria is generally greatly reduced by the freeze-drying process.

Typically, a study was conducted to confirm the effect of storage temperature and polymer on the stability of lactic acid bacteria by freeze-drying Lactobacillus rhamnosus coated with gelatin (Claude P. C. et al, Food. Res. Int., 1996, 29, 555^{˜}562). However, the above study discloses that about 1% of lactic acid bacteria survived after 6 months of storage at 20° C., and only about 0.2% of lactic acid bacteria survived after 12 months; therefore, there is a need to develop methods that can increase the survival rate and storage stability of lactic acid bacteria

Meanwhile, methods of coating lactic acid bacteria using alginate capsules have been known, but alginate is unstable to acid and heat. To this end, according to the conventional methods of producing capsules using alginate, they are produced by spraying a mixture of alginate and microorganisms into a coagulation solution such as a CaCl₂ solution. Thus, hardening begins when the alginate reacts with the calcium ions of the coagulation solution from the particle surface of the sprayed mixture of alginate and microorganisms, and sufficient calcium ions cannot move into the particles in a short period of time. Accordingly, it is difficult to mass-produce alginate capsules that have firmly and densely hardened even to the inside of alginate at high yield.

### DISCLOSURE

### Technical Problem

The present inventors have developed a process for producing lactic acid bacteria encapsulated with calcium-alginate beads, which increases the production yield of beads and improves the survival rate after freeze-drying and stability, thereby completing the present disclosure.

### Technical Solution

It is one object of the present disclosure to provide a method for producing lactic acid bacteria encapsulated with calcium-alginate beads.

It is another object of the present disclosure to provide a lactic acid bacterium encapsulated with calcium-alginate beads produced by the above method.

It is still another object of the present disclosure to provide a method for improving the production yield of calcium-alginate beads for encapsulating lactic acid bacteria.

It is yet another object of the present disclosure to provide a method for improving the survival rate of freeze-dried lactic acid bacteria.

### Advantageous Effects

The method for producing lactic acid bacteria encapsulated with calcium-alginate beads of the present disclosure increases the production yield of the beads and can provide lactic acid bacteria with improved survival rate after freeze-drying and stability.

### BRIEF DESCRIPTION OF DRAWINGS

FIG. 1 is a schematic diagram showing the method for producing lactic acid bacteria encapsulated with calcium-alginate beads of the present disclosure.

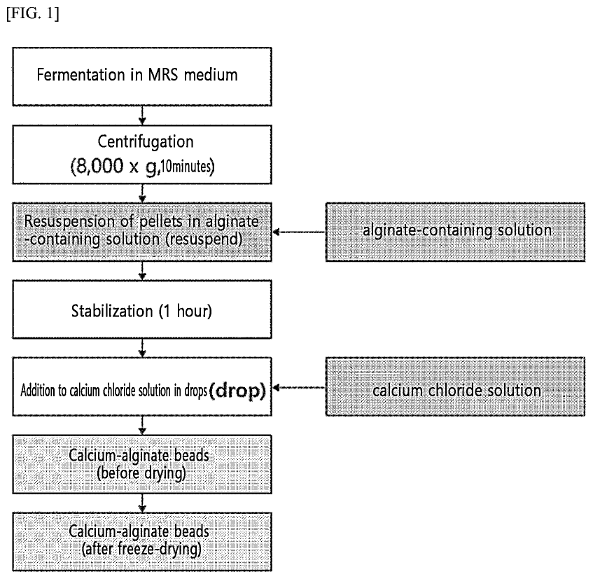

FIG. 2 is a diagram showing the results of evaluating stability in the gastrointestinal tract according to the presence or absence of calcium-alginate beads.

### DETAILED DESCRIPTION OF PREFERRED EMBODIMENTS

The present disclosure will be described in detail as follows. Meanwhile, each description and embodiment disclosed herein can be applied to other descriptions and embodiments, respectively. That is, all combinations of various elements disclosed herein fall within the scope of the present disclosure. Further, the scope of the present disclosure is not limited by the specific description described below.

Additionally, those of ordinary skill in the art may be able to recognize or confirm, using only conventional experimentation, many equivalents to the particular aspects of the disclosure described herein. Furthermore, it is also intended that these equivalents be included in the present disclosure.

One aspect of the present disclosure provides a method for producing lactic acid bacteria encapsulated with calcium-alginate beads, comprising:
(a) culturing lactic acid bacteria and recovering bacterial cells;
(b) mixing the lactic acid bacteria of step (a) with an alginate-containing solution; and
(c) adding the mixture of step (b) to a calcium-containing solution to form calcium-alginate beads,
wherein the alginate-containing solution comprises sucrose, sorbitol, or soy peptone, and the calcium-containing solution comprises trehalose or maltodextrin.

In one embodiment, the method may further comprise (d) freeze-drying the lactic acid bacteria encapsulated with the calcium-alginate beads of step (c), but is not limited thereto.

As used herein, the term “freeze-drying” refers to a method in which a material to be dried is frozen by rapidly lowering the temperature of a container, then the internal pressure of the container is made into a vacuum, and the solidified solvent contained in the material is immediately sublimated into vapor to perform drying. Freeze-drying is a method by which damage to a substance sensitive to heat can be minimized and the substance can be preserved for a long time, and is useful in terms of contamination prevention, storage, transportation, and economic efficiency. However, during the freeze-drying process of lactic acid bacteria, the activity and survival rate of the lactic acid bacteria are rapidly reduced, ice particles are formed during freezing, and thus there is a problem in that the membrane structure of the lactic acid bacteria cells is damaged. A substance which is added together during freeze-drying so that the function can be recovered during rehydration without damaging or killing the lactic acid bacteria, in order to solve such a problem, is referred to as a “cryoprotectant”, which serves to impart physicochemical stability to the lactic acid bacteria to increase the survival rate.

In the present disclosure, the freezing temperature of the freeze-drying may be a sub-zero temperature, for example, −10° C. to −196° C. (boiling point of liquid nitrogen), −40° C. to −196° C., −50° C. to −196° C., and −70° C. to −196° C., and at such low temperatures, all biological activities of lactic acid bacteria including biochemical reactions that lead to cell death are effectively stopped.

As used herein, the term “cryoprotecting” means protecting lactic acid bacteria tissue from being frozen, when the lactic acid bacteria are freeze-dried and then preserved in order to maintain the activity thereof as it is.

For the purpose of the present disclosure, the lactic acid bacteria encapsulated with calcium-alginate beads produced by the method for producing lactic acid bacteria encapsulated with calcium-alginate beads above may have the improved survival rate after freeze-drying and stability.

Hereinafter, the method of producing lactic acid bacteria encapsulated with calcium-alginate beads of the present disclosure will be described in detail.

First, (a) lactic acid bacteria are cultured and the bacterial cells are recovered.

As used herein, the term “lactic acid bacteria” is a generic term for bacteria which acquire energy by fermenting saccharides and produce a large amount of lactic acid. Although not particularly limited thereto, the lactic acid bacteria may comprise at least one selected from the group consisting of Lactobacillus sp., Bifidobacterium sp., Streptococcus sp., Lactococcus sp., Enterococcus sp., Pediococcus sp., Leuconostoc sp., and Weissella sp, but is not limited thereto.

Specifically, the lactic acid bacteria may comprise at least one selected from the group consisting of Lactobacillus plantarum, Lactobacillus casei, Lactobacillus rhamnosus, Lactobacillus acidophilus, Bifidobacterium bifidum, Bifidobacterium longum, Bifidobacterium breve, Streptococcus faecalis, and Lactococcus lactis subsp. lactis, but is not limited thereto.

More specifically, the lactic acid bacteria may comprise at least one selected from the group consisting of Lactobacillus plantarum CJLP133, Lactobacillus plantarum CJLP243, Lactobacillus plantarum CJLP136, Lactobacillus plantarum CJLP55, and Lactobacillus plantarum CJLP56, but is not limited thereto.

The strains are deposited at the gene bank of Korea Research Institute of Bioscience and Biotechnology and can be easily obtained therefrom.

As used herein, the term “cultivation” means that the lactic acid bacteria are grown under appropriately controlled environmental conditions. The cultivation process of the present disclosure may be performed in a suitable culture medium and culture conditions known in the art. Such a cultivation process may be easily adjusted for use by those skilled in the art according to the strain to be selected. Specifically, the cultivation may be a batch culture, a continuous culture, and a fed-batch culture, but is not limited thereto.

As used herein, the term “medium” refers to a mixture of materials which contains nutrient materials required for the cultivation of the microorganism of the present disclosure as a main ingredient, and it supplies nutrient materials and growth factors, along with water that is essential for survival and growth. Specifically, the medium and other culture conditions used for culturing the microorganism of the present disclosure may be any medium used for conventional cultivation of microorganisms without any particular limitation. However, the microorganism of the present disclosure may be cultured under aerobic conditions in a conventional medium containing an appropriate carbon source, nitrogen source, phosphorus source, inorganic compound, amino acid, and/or vitamin, etc., while adjusting temperature, pH, etc.

In the present disclosure, the carbon source may include carbohydrates, such as glucose, fructose, sucrose, maltose, etc.; sugar alcohols, such as mannitol, sorbitol, etc.; alcohols, such as glycerol, etc.; organic acids, such as pyruvic acid, lactic acid, citric acid, etc.; amino acids, such as glutamic acid, methionine, lysine, etc. Additionally, the carbon source may include natural organic nutrients such as starch hydrolysate, molasses, blackstrap molasses, rice bran, cassava, sugar cane molasses, and corn steep liquor, etc. Specifically, carbohydrates such as glucose and sterilized pretreated molasses (i.e., molasses converted to reducing sugar) may be used, and in addition, various other carbon sources in an appropriate amount may be used without limitation. These carbon sources may be used alone or in a combination of two or more kinds, but are not limited thereto.

The nitrogen source may include inorganic nitrogen sources, such as ammonia, ammonium sulfate, ammonium chloride, ammonium acetate, ammonium phosphate, ammonium carbonate, ammonium nitrate, etc.; amino acids, such as glutamic acid, methionine, glutamine, etc.; and organic nitrogen sources, such as peptone, NZ-amine, meat extract, yeast extract, malt extract, corn steep liquor, casein hydrolysate, fish or decomposition product thereof, defatted soybean cake or decomposition product thereof, etc. These nitrogen sources may be used alone or in a combination of two or more kinds, but are not limited thereto.

The phosphorus source may include monopotassium phosphate, dipotassium phosphate, or corresponding sodium-containing salts, etc. Examples of the inorganic compounds may include sodium chloride, calcium chloride, iron chloride, magnesium sulfate, iron sulfate, manganese sulfate, calcium carbonate, etc. Additionally, amino acids, vitamins, and/or appropriate precursors, etc., may be included. These constituting ingredients or precursors may be added to a medium in a batch or continuous manner, but is not limited thereto.

In the present disclosure, the pH of the medium may be adjusted by adding a compound such as ammonium hydroxide, potassium hydroxide, ammonia, phosphoric acid, sulfuric acid, etc. during the cultivation of lactic acid bacteria in an appropriate manner. In addition, bubble formation may be prevented during the cultivation using an antifoaming agent such as fatty acid polyglycol ester. Further, in order to maintain aerobic conditions of the medium, oxygen gas or a gas containing oxygen may be injected to the medium; or to maintain anaerobic or microaerobic conditions, the cultivation may be performed without injection of any gas to the medium, or nitrogen gas, hydrogen gas, or carbon dioxide may be injected to the medium, but is not limited thereto.

The temperature of the medium may be in the range from 20° C. to 50° C., specifically from 30° C. to 37° C., but is not limited thereto. Additionally, the cultivation may be continued until a desired amount of useful materials is obtained, specifically for 10 hours to 100 hours, but is not limited thereto.

In the step of recovering lactic acid bacterial cells, the desired bacterial cells may be collected using a suitable method known in the art according to the method of culturing lactic acid bacteria of the present disclosure, for example, a batch culture, continuous culture, or fed-batch culture method. For example, methods such as centrifugation, filtration, treatment with a protein crystallizing precipitant (salting-out method), extraction, ultrasonic disruption, ultrafiltration, dialysis, various kinds of chromatographies such as molecular sieve chromatography (gel filtration), adsorption chromatography, ion exchange chromatography, affinity chromatography, etc., HPLC or a combination thereof may be used, but are not limited to these examples.

The recovering step may comprise an additional purification step, which may be performed using an appropriate method known in the art, and the recovered lactic acid bacterial cells may be purified.

Subsequently, (b) the lactic acid bacteria of step (a) are collected using methods such as centrifugation, and then mixed with an alginate-containing solution.

As used herein, the term “alginic acid or alginate” is a polymer of two types of uronic acids with a degree of polymerization of 80 and a molecular weight of about 1,500, and has an unbranched structure in which hundreds of D-mannuronic acid (hereinafter referred to as M) and L-guluronic acid are linked through β-1, 4 bonds.

In one embodiment of the present disclosure, in order to increase the cryoprotective effect and thus increase the survival rate of lactic acid bacteria encapsulated with calcium-alginate beads after freeze-drying, they were prepared by adding saccharides and proteins to an alginate-containing solution. Frequently used cryoprotectant for proteins typically includes skim milk, but it was confirmed that when skim milk was added, the calcium component of skim milk first reacted with alginate and became hardened. Thus, it was excluded from the experimental group since beads were not produced properly.

Specifically, the alginate-containing solution may comprise sucrose, sorbitol, or soy peptone, and more specifically, soy peptone, but is not limited thereto.

As used herein, the term “soy peptone” refers to a protein derived from soybeans, which is mainly used as a nutrient source in microbial media. The protein content of isolated soy protein is about 90%, and globulin is most abundantly included in soy peptone.

In one embodiment, the alginate-containing solution comprises sucrose, sorbitol or soy peptone, more specifically 5% to 10% by weight, more specifically 6% to 8% by weight, and even more specifically about 7% by weight of soy peptone, but is not limited thereto.

When the sucrose, sorbitol, or soy peptone is comprised in the above weight percent, the production yield of beads increases, and it can serve as a cryoprotectant to prevent damage or death of lactic acid bacteria due to freeze-drying and to increase stability. When sucrose, sorbitol, or soy peptone is comprised in a weight percent higher than the optimal range, the bead formation of alginate is hindered, and thus the beads are not formed densely, which reduces the protective effect by gastric acid.

In one embodiment, the alginate-containing solution is prepared by dissolving sodium alginate in an amount of 1% to 3% by weight, more specifically about 2% by weight, and may additionally include trehalose or maltodextrin, but is not limited thereto.

When the sodium alginate is comprised in a weight percent lower than the optimal range, the hardening of the beads may be weak, leading to elution of the lactic acid bacteria and cryoprotective ingredients, and when the sodium alginate is comprised in a weight percent higher than the optimal range, the hardening of the beads may be too strong that the lactic acid bacteria may not come out easily when they are consumed afterward, or the bacteria may die, which is a problem.

As used herein, the term “trehalose” is a saccharide which is widely present in nature such as plants or microorganisms, etc. For the purpose of the present disclosure, the trehalose comprised in the alginate-containing solution may act as a cryoprotectant which prevents damage or death of the lactic acid bacteria caused by freeze-drying and helps recover the function thereof during rehydration, thereby increasing the cryoprotective effect of the lactic acid bacteria.

As used herein, the term “maltodextrin” is a white powder based on porous particles, and is a food additive often used in general foods such as yogurt, sauce, salad dressings, etc. For the purpose of the present disclosure, the maltodextrin comprised in the alginate-containing solution may be used as a porous support during freeze-drying of lactic acid bacteria and act as a cryoprotectant which prevents damage or death of the lactic acid bacteria caused by freeze-drying. The porous support is based in the form of porous particles and serves to block the inflow of external moisture and air.

In one embodiment, the trehalose may be comprised in an amount of 5% to 10% by weight, more specifically 6% to 8% by weight, and even more specifically about 7% by weight, and the maltodextrin may be comprised in an amount of 5% to 10% by weight, more specifically 6% to 8% by weight, and even more specifically about 6% by weight, but is not limited thereto.

In one embodiment, the ratio of the weight of the alginate-containing solution and the weight of lactic acid bacteria in the mixture of step (b) may be 1:1 to 10:1, more specifically 1:1 to 3:1, and more specifically about 2:1, but is not limited thereto.

As the solid weight of lactic acid bacteria is comprised at the optimal ratio, the production cost can be maintained at an appropriate level while maintaining the cryoprotective effect and the yield of dried lactic acid bacteria powder when using the process. When the ratio of the weight of the alginate-containing solution and the weight of lactic acid bacteria deviates from the optimal ratio, there is a problem in that the production yield of lactic acid bacteria per unit time and/or per kg produced decreases, thereby lowering process efficiency.

Additionally, flowability (viscosity) can be maintained by comprising the total solid content of the alginate-containing solution at 20% to 30% by weight.

In one embodiment, a prebiotic may be optionally further added during mixing in step (b). Specifically, a prebiotic may be added when the lactic acid bacteria are mixed with the coating agent. The prebiotic serves as food for the lactic acid bacteria. The prebiotic may be any one or more selected from fructooligosaccharides, galactooligosaccharides, maltitol, lactitol, and inulin, and specifically, fructooligosaccharide or inulin. The prebiotic may be added in an amount of 0.1% to 5% by weight, based on the weight of the lactic acid bacteria.

Subsequently, (c) the mixture of step (b) is added to a calcium-containing solution to form calcium-alginate beads.

Specifically, the step (c) is an extrusion step. When the mixture of step (b) is added to a calcium-containing solution in droplets or by spraying, thereby allowing it to react with the calcium-containing solution, a matrix is formed through alginate-calcium crosslinking. As a result, the lactic acid bacteria are encapsulated in beads within the calcium-containing solution, and alginate-calcium beads are formed, but is not limited thereto.

The formation of bead particles through the alginate-calcium reaction is carried out at a temperature under room temperature (e.g., 25° C.). Since this reaction involves less physical stress, there is little impact on the survival rate of the living bacteria. The alginate-calcium bead particles formed by extrusion show pH-dependent release; thus, the alginate-calcium bead particles are not decomposed under acidic conditions, such as gastric acid, and are slowly decomposed in a neutral environment, such as the intestinal environment, greatly contributing to an improvement in the survival rate of lactic acid bacteria in the intestine.

Saccharides, proteins, lipids, etc. may be used as cryoprotective ingredients. When proteins are added to a calcium-containing solution, the amphipathic nature of amino acids prevents alginate from combining with calcium ions, preventing the formation of alginate beads with a dense structure. Lipids do not mix well with water and are not suitable for use in calcium-containing solutions. Therefore, in one embodiment of the present disclosure, it was confirmed that the addition of saccharides to a calcium-containing solution improved the production yield and survival rate of alginate beads.

Specifically, the calcium-containing solution may comprise trehalose or maltodextrin, and more specifically, trehalose, but is not limited thereto.

When trehalose or maltodextrin is comprised, the production yield of the beads increases and can serve as a cryoprotectant to prevent damage or death of lactic acid bacteria caused by freeze-drying and increase stability.

In one embodiment, in order to establish the calcium-containing solution to a concentration similar to the solid content of the alginate-containing solution mixed with lactic acid bacteria pellets, the calcium-containing solution may comprise trehalose or maltodextrin, more specifically, trehalose in an amount of 15% to 35% by weight, even more specifically, 20% to 30% by weight, and further more specifically about 25% by weight, but is not limited thereto.

By establishing the concentrations of the mixture in step (b) and the calcium-containing solution to be similar, dissolution of the cryoprotective ingredients of the solution containing lactic acid bacteria and alginic acid into the calcium-containing solution after being added in droplets may be prevented as much as possible.

In one embodiment, the calcium-containing solution may be one in which calcium chloride is dissolved in water at 0.5% to 1.5% by weight, more specifically at about 1% by weight, but is not limited thereto.

When the calcium chloride is included in a weight percent lower than the optimal range, the bead structure may not be produced densely and the cryoprotective effect may not be sufficient, and when the calcium chloride is included in a weight percent higher than the optimal range, the hardening of the beads may be too strong, thus, there is a problem in commercialization due to insufficient elution of lactic acid bacteria in the final products.

Specifically, after step (c), a step of storing the solution containing the alginate-calcium beads at a temperature of 4° C. to 20° C. for 30 to 60 minutes may be further comprised.

This step is a maturation process. As sodium ions are replaced with calcium ions, alginate forms a network structure and encapsulation is carried out. Through this maturation process, the density of the particle structure may be increased.

The method of the present disclosure may further comprise adding one or more types, specifically, one type, two types, or three types, selected from the group consisting of a cryoprotectant, a porous support, and a nitrogen source.

Specifically, the method of the present disclosure may further comprise adding an ingredient known as a conventional cryoprotectant, excluding the alginate-containing solution and the calcium-containing solution. That is, as used herein, the term “cryoprotectant”, which is further added to produce lactic acid bacteria encapsulated with calcium-alginate beads, refers to a substance having cryoprotective efficacy commonly used in the technical field to which the present disclosure belongs. As the cryoprotectant, commercially available products can be purchased and used, and the type thereof is not particularly limited. Specifically, the cryoprotectant may be trehalose, saccharide, amino acid, peptide, gelatin, glycerol, sugar alcohol, whey, alginic acid, ascorbic acid, yeast extract, skim milk, etc., but is not limited thereto.

As used herein, the “porous support” serves to block the inflow of external moisture and air and impart porosity to freeze-dried lactic acid bacteria to facilitate rehydration. The porous support is a porous support commonly used during freeze-drying in the technical field to which the present disclosure belongs. As the porous support, commercially available products can be purchased and used, and the type thereof is not particularly limited. The porous support may be specifically maltodextrin, alginate, chitosan, starch, polyethylene glycol, propylene glycol, triacetin, acetyl triethyl citrate, triethyl citrate, glycerin, or a combination thereof, but is not limited thereto.

As used herein, the term “nitrogen source (N-source)” refers to a substance used as a nitrogen energy source for lactic acid bacteria, and serves to prevent damage to bacterial cells caused by post-fermentation. When lactic acid bacteria are mixed with a composition for cryoprotection, lactic acid bacteria, which live in the absence of an energy source, generate an organic acid, causing a decrease in pH, and thus inducing the death of lactic acid bacteria. Accordingly, the nitrogen energy source prevents the generation of organic acids and the resulting pH decrease, and thus, the death of lactic acid bacteria can be prevented.

The nitrogen source is a nitrogen source commonly used during freeze-drying in the technical field to which the present disclosure belongs. As the nitrogen source, commercially available products can be purchased and used, and the type thereof is not particularly limited. The nitrogen source may be skimmed milk powder, whey protein, yeast extract, malt extract, beef extract, casein hydrolyzate, malt extract, tryptone, cysteine, peptone, etc., and in particular, the peptone may be soy peptone, fish peptone, proteose peptone, casein peptone, peptone No. 3, etc.

Subsequently, (d) freeze-drying the lactic acid bacteria encapsulated with the calcium-alginate beads of step (c) may be further added. The freeze-drying step may be carried out by transferring the alginate-calcium beads containing lactic acid bacteria produced in step (c) to a freeze-drying tray while increasing the temperature from −20° C. to 30° C. for 50 hours.

Another aspect of the present disclosure provides lactic acid bacteria encapsulated with calcium-alginate beads produced by the method described above.

The method and the lactic acid bacteria encapsulated with calcium-alginate beads are as described above.

Specifically, the lactic acid bacteria may have improved survival rate after freeze-drying compared to that of a lactic acid bacterium which is not encapsulated with calcium-alginate beads, but are not limited thereto.

Specifically, the lactic acid bacteria may have improved stability compared to that of a lactic acid bacterium which is not encapsulated with calcium-alginate beads, but are not limited thereto.

For the purpose of the present disclosure, the term “stability” may refer to storage (distribution) stability or stability in the gastrointestinal tract, but is not limited thereto.

Specifically, it may refer to high-temperature stability, acid resistance, or bile resistance, etc.

Still another aspect of the present disclosure provides a method for improving the production yield of calcium-alginate beads for encapsulating lactic acid bacteria, comprising:
(a) culturing lactic acid bacteria and recovering bacterial cells;
(b) mixing the lactic acid bacteria of step (a) with an alginate-containing solution; and
(c) adding the mixture of step (b) to a calcium-containing solution to form calcium-alginate beads,
wherein the alginate-containing solution comprises sucrose, sorbitol, or soy peptone, and the calcium-containing solution comprises trehalose or maltodextrin.

The method and the lactic acid bacteria encapsulated with calcium-alginate beads are as described above.

Yet another aspect of the present disclosure provides a method for improving the survival rate of freeze-dried lactic acid bacteria, comprising:
(a) culturing lactic acid bacteria and recovering bacterial cells;
(b) mixing the lactic acid bacteria of step (a) with an alginate-containing solution;
(c) adding the mixture of step (b) to a calcium-containing solution to form calcium-alginate beads; and
(d) freeze-drying the lactic acid bacteria encapsulated with the calcium-alginate beads of step (c),
wherein the alginate-containing solution comprises sucrose, sorbitol, or soy peptone, and the calcium-containing solution comprises trehalose or maltodextrin.

The method and the lactic acid bacteria encapsulated with calcium-alginate beads are as described above.

Even another aspect of the present disclosure provides lactic acid bacteria encapsulated with calcium-alginate beads and freeze-dried, which are produced by the method above.

The method and the lactic acid bacteria encapsulated with calcium-alginate beads are as described above.

Specifically, the lactic acid bacteria may have improved survival rate after freeze-drying compared to that of a lactic acid bacterium which is not encapsulated with calcium-alginate beads, but are not limited thereto.

Specifically, the lactic acid bacteria may have improved stability compared to that of a lactic acid bacterium which is not encapsulated with calcium-alginate beads, but are not limited thereto.

### MODE FOR CARRYING OUT THE INVENTION

Hereinafter, the present disclosure will be described in detail by way of Examples. However, these Examples are merely preferred Examples given for illustrative purposes, and thus, the scope of the present disclosure is not intended to be limited by these Examples.

### Comparative Example 1. Preparation of Freeze-Dried Powder Comprising Lactic Acid Bacteria

### Comparative Example 1-1. Cultivation of Lactic Acid Bacteria

Lactobacillus plantarum CJLP133 was cultured in MRS liquid medium (Difco, USA) at 37° C. for 10 hours. The number of lactic acid bacteria in the culture medium was confirmed to be approximately 8.68×10\hat{ }9 CFU/mL. MRS medium components were prepared as follows: Proteose Peptone 10 g, Beef Extract 10 g, Yeast Extract 5 g, Dextrose 20 g, Polysorbate 80 1 g, Ammonium Citrate 2 g, Sodium Acetate 5 g, Magnesium Sulfate 0.1 g, Manganese Sulfate 0.05 g, and Dipotassium Phosphate 2 g were mixed in 1 L of distilled water, dissolved and sterilized.

### Comparative Example 1-2. Preparation of Latic Acid Solution Comprising Cryoprotectant

The culture medium, in which the lactic acid bacteria of Comparative Example 1-1 were cultured, was centrifuged for 10 minutes, the supernatant was discarded, and a pellet was obtained. The thus-obtained bacterial cells were stabilized by stirring for 30 minutes with a sterilized cryoprotectant at a ratio (w/w) of 1:4. The cryoprotectant solution was prepared by mixing Maltodextrin 140 g, Trehalose 140 g, Skim milk 30 g, and Distilled water 690 g, followed by sterilization.

### Comparative Example 1-3. Freeze-Drying of Lactic Acid Bacteria Comprising Cryoprotectant

The lactic acid bacteria of Comparative Example 1-2 comprising the cryoprotectant was rapidly frozen and then dried using a freeze dryer. Freeze-drying was carried out by raising the temperature from −20° C. to 30° C. for 50 hours.

### Example 1. Preparation of Lactic Acid Bacteria Encapsulated with Calcium-Alginate Beads Using Calcium-Containing Solution Comprising Calcium Chloride and Freeze-Drying Thereof

### Example 1-1. Cultivation of Lactic Acid Bacteria

Lactobacillus plantarum CJLP133 was cultured in MRS liquid medium (Difco, USA) at 37° C. for 10 hours. The number of lactic acid bacteria in the culture medium was confirmed to be approximately 8.68×10\hat{ }9 CFU/mL. MRS medium components were prepared as follows: Proteose Peptone 10 g, Beef Extract 10 g, Yeast Extract 5 g, Dextrose 20 g, Polysorbate 80 1 g, Ammonium Citrate 2 g, Sodium Acetate 5 g, Magnesium Sulfate 0.1 g, Manganese Sulfate 0.05 g, and Dipotassium Phosphate 2 g were mixed in 1 L of distilled water, dissolved and sterilized.

### Example 1-2. Preparation of Sodium Alginate Solution Comprising Lactic Acid Bacteria

The culture medium, in which the lactic acid bacteria of Example 1-1 were cultured, was centrifuged for 10 minutes, the supernatant was discarded, and a pellet was obtained. The alginate-containing solution (alginate solution) was prepared by mixing the composition shown in Table 1 below, followed by sterilization. The thus-obtained bacterial cells were stabilized by stirring for 30 minutes with the sterilized sodium alginate solution at a ratio (w/w) of 1:2.

TABLE 1

| | | |
|---|---|---|
| Sodium alginate | 20 | g |
| Trehalose | 140 | g |
| Dextrin | 60 | g |
| Distilled water | 780 | g |

### Example 1-3. Preparation of Lactic Acid Bacteria Encapsulated with Calcium-Alginate Beads

The calcium-containing solution was prepared by mixing the composition shown in Table 2 below, followed by sterilization.

Calcium-alginate beads were prepared by adding the mixture of Example 1-2 to the calcium-containing solution in droplets using a syringe. Thereafter, the resultant was immersed for 1 hour, and as sodium ions were replaced with calcium ions, alginate formed a network structure, thereby allowing encapsulation.

TABLE 2

| | | |
|---|---|---|
| CaCl₂ | 10 | g |
| Distilled water | 990 | g |

### Example 1-4. Freeze-Drying

The lactic acid bacteria encapsulated with the calcium-alginate beads of Example 1-3 were rapidly frozen and then dried using a freeze dryer. Freeze-drying was carried out by raising the temperature from −20° C. to 30° C. for 50 hours.

### Example 2. Preparation of Lactic Acid Bacteria Encapsulated with Calcium-Alginate Beads Using Calcium-Containing Solution Comprising Calcium Chloride and Maltodextrin, and Freeze-Drying Thereof

Lactic acid bacteria were prepared in the same manner as in Example 1, except that a calcium-containing solution with the composition shown in Table 3 below was used in Example 1-3.

TABLE 3

| | | |
|---|---|---|
| CaCl₂ | 10 | g |
| Maltodextrin | 250 | g |
| Distilled water | 740 | g |

### Example 3. Preparation of Lactic Acid Bacteria Encapsulated with Calcium-Alginate Beads Using Calcium-Containing Solution Comprising Calcium Chloride and Trehalose, and Freeze-Drying Thereof

Lactic acid bacteria were prepared in the same manner as Example 1, except that a calcium-containing solution with the composition shown in Table 4 below was used in Example 1-3.

TABLE 4

| | | |
|---|---|---|
| CaCl₂ | 10 | g |
| Trehalose | 250 | g |
| Distilled water | 740 | g |

### Example 4. Preparation of Lactic Acid Bacteria Encapsulated with Calcium-Alginate Beads Using Alginate-Containing Solution Comprising Sucrose and Freeze-Drying Thereof

Lactic acid bacteria were prepared in the same manner as Example 1, except that an alginate-containing solution with the composition shown in Table 5 below was used in Example 1-2, and a calcium-containing solution with the composition shown in Table 6 below was used in Example 1-3.

TABLE 5

| | | |
|---|---|---|
| Sodium alginate | 20 | g |
| Trehalose | 70 | g |
| Sucrose | 70 | g |
| Maltodextrin | 60 | g |
| Distilled water | 780 | g |

TABLE 6

| | | |
|---|---|---|
| CaCl₂ | 10 | g |
| Trehalose | 250 | g |
| Distilled water | 740 | g |

### Example 5. Preparation of Lactic Acid Bacteria Encapsulated with Calcium-Alginate Beads Using Alginate-Containing Solution Comprising Sorbitol and Freeze-Drying Thereof

Lactic acid bacteria were prepared in the same manner as Example 1, except that an alginate-containing solution with the composition shown in Table 7 below was used in Example 1-2, and a calcium-containing solution with the composition shown in Table 6 above was used in Example 1-3.

TABLE 7

| | | |
|---|---|---|
| Sodium alginate | 20 | g |
| Trehalose | 70 | g |
| Sorbitol | 70 | g |
| Maltodextrin | 60 | g |
| Distilled water | 780 | g |

### Example 6. Preparation of Lactic Acid Bacteria Encapsulated with Calcium-Alginate Beads Using Alginate-Containing Solution Comprising Soy Peptone and Freeze-Drying Thereof

Lactic acid bacteria were prepared in the same manner as Example 1, except that an alginate-containing solution with the composition shown in Table 8 below was used in Example 1-2, and a calcium-containing solution with the composition shown in Table 6 above was used in Example 1-3.

TABLE 8

| | | |
|---|---|---|
| Sodium alginate | 20 | g |
| Trehalose | 70 | g |
| Soy Peptone | 70 | g |
| Maltodextrin | 60 | g |
| Distilled water | 780 | g |

### Experimental Example 1. Measurement of Production Yield of Calcium-Alginate Beads and Survival Rate of Lactic Acid Bacteria after Freeze-Drying According to Compositions of Calcium-Containing Solution

An attempt was made to measure the production yield of the calcium-alginate beads of Examples 1 to 3 and the survival rate of lactic acid bacteria after freeze-drying.

The method for calculating the production yield of calcium-alginate beads is as follows.

Specifically, the alginate-containing solution mixed with the lactic acid bacteria pellets of Examples 1 to 3 was diluted to an appropriate concentration and smeared on MRS agar medium to measure the number of bacteria (cfu/g). Additionally, the prepared beads were also diluted to an appropriate concentration and smeared on MRS agar medium at an appropriate concentration to measure the number of bacteria (cfu/g). In order to calculate the production yield, the total number of bacteria was calculated by multiplying the measured number of bacteria by each of the weight of the added alginate-containing solution and the weight of the prepared beads. The production yield (before drying) of the calcium-alginate beads was measured by comparing the measured total number of bacteria.

In order to calculate the survival rate of the lactic acid bacteria of Examples 1 to 3 after freeze-drying, the total number of bacteria was calculated.

Specifically, the weight of the beads of Examples 1-3 after freeze-drying was measured, and the beads were diluted in MRS agar medium to an appropriate concentration and smeared to measure the number of bacteria (cfu/g). The calculated total number of bacteria was compared with the total number of bacteria in the alginate-containing solution calculated above to measure the survival rate of lactic acid bacteria encapsulated with calcium-alginate beads after freeze-drying. The results are shown in Tables 9 to 11 below, respectively.

TABLE 9

| | | | Survival Rate after |
|---|---|---|---|
| | L. plantarum | Production | Freeze-Drying |
| | alginate | Yield | (After freezing |
| Example 1 | solution | (Before drying) | drying) |
| Visible cells (cfu/g) | 1.00.E+11 | 6.07.E+10 | 8.65.E+10 |
| Sol or Bead (g) | 210.0 | 201.4 | 17.9 |
| Total cells (cfu) | 2.10.E+13 | 1.22.E+13 | 1.55.E+12 |
| Survival ratio (%) | — | 58.21 | 7.38 |

TABLE 10

| | | | Survival Rate after |
|---|---|---|---|
| | L. plantarum | Production | Freeze-Drying |
| | alginate | Yield | (After freezing |
| Example 2 | solution | (Before drying) | drying) |
| Visible cells (cfu/g) | 1.00.E+11 | 9.05.E+10 | 8.52.E+10 |
| Sol or Bead (g) | 210.0 | 207.2 | 47.7 |
| Total cells (cfu) | 2.10.E+13 | 1.88.E+13 | 4.06.E+12 |
| Survival ratio (%) | — | 89.29 | 19.33 |

TABLE 10

| | | | Survival Rate after |
|---|---|---|---|
| | L. plantarum | Production | Freeze-Drying |
| | alginate | Yield | (After freezing |
| Example 3 | solution | (Before drying) | drying) |
| Visible cells (cfu/g) | 1.00.E+11 | 9.89.E+10 | 2.03.E+11 |
| Sol or Bead (g) | 210.0 | 206.1 | 47.4 |
| Total cells (cfu) | 2.10.E+13 | 2.04.E+13 | 9.60.E+12 |
| Survival ratio (%) | — | 97.07 | 45.73 |

With reference to Table 9 above, when the CaCl₂) solution consisting only of CaCl₂) of Example 1 was used, the production yield of beads was 58.21%, and the final survival rate after freeze-drying was very low at 7.38%.

With reference to Table 10 above, when the CaCl₂) solution containing 25% maltodextrin of Example 2 was used, the production yield of beads was 89.29% and the final survival rate after freeze-drying was 19.33%, confirming that the results were improved compared to Example 1. This suggests that the production yield of beads was greatly improved by 25% maltodextrin, which has a similar concentration to the alginate-containing solution, and the survival rate after freeze-drying was also improved accordingly.

With reference to Table 11 above, when the CaCl₂) solution containing 25% trehalose of Example 3 was used, the production yield of beads was 97.07% and the final survival rate after freeze-drying was 45.73%, confirming that the results were further improved.

### Experimental Example 2. Measurement of Production Yield of Calcium-Alginate Beads and Survival Rate of Lactic Acid Bacteria after Freeze-Drying According to Compositions of Alginate-Containing Solution

In the same manner as in Experimental Example 1, the production yield of the calcium-alginate beads of Examples 4 to 6 and the survival rate of lactic acid bacteria after freeze-drying were measured, and the results are shown in Tables 12 to 14 below.

TABLE 12

| | | | Survival Rate after |
|---|---|---|---|
| | L. plantarum | Production | Freeze-Drying |
| | alginate | Yield | (After freezing |
| Example 4 | solution | (Before drying) | drying) |
| Visible cells (cfu/g) | 8.90.E+10 | 1.05.E+11 | 1.86.E+11 |
| Sol or Bead (g) | 210.0 | 168.8 | 50.8 |
| Total cells (cfu) | 1.87.E+13 | 1.77.E+13 | 9.44.E+12 |
| Survival ratio (%) | — | 94.86 | 50.52 |

TABLE 13

| | | | Survival Rate after |
|---|---|---|---|
| | L. plantarum | Production | Freeze-Drying |
| | alginate | Yield | (After freezing |
| Example 5 | solution | (Before drying) | drying) |
| Visible cells (cfu/g) | 8.90.E+10 | 1.02.E+11 | 2.58.E+11 |
| Sol or Bead (g) | 210.0 | 167.5 | 49.5 |
| Total cells (cfu) | 1.87.E+13 | 1.71.E+13 | 1.28.E+13 |
| Survival ratio (%) | — | 91.42 | 68.31 |

TABLE 14

| | | | Survival Rate after |
|---|---|---|---|
| | L. plantarum | Production | Freeze-Drying |
| | alginate | Yield | (After freezing |
| Example 6 | solution | (Before drying) | drying) |
| Visible cells (cfu/g) | 1.00.E+11 | 1.10.E+11 | 3.80.E+11 |
| Sol or Bead (g) | 210.0 | 188.5 | 47.7 |
| Total cells (cfu) | 2.10.E+13 | 2.07.E+13 | 1.81.E+13 |
| Survival ratio (%) | — | 98.72 | 86.32 |

With reference to Table 12 above, when the alginate-containing solution comprising sucrose of Example 4 was used, the production yield of beads was 94.3% and the final survival rate after freeze-drying was 50.44%, which is similar to Example 3.

With reference to Table 13 above, when the alginate-containing solution comprising sorbitol of Example 5 was used, the production yield of beads was 91.49% and the final survival rate after freeze-drying was 68.36%, confirming that the results were improved compared to Example 4.

With reference to Table 14 above, when the alginate-containing solution comprising soy peptone of Example 6 was used, the production yield of beads was 98.77% and the final survival rate after freeze-drying was 86.35%, confirming that the results were greatly improved. Accordingly, the optimal composition that can maximize the production yield of beads and cryoprotective effect of lactic acid bacteria was confirmed.

### Experimental Example 3. Evaluation of High-Temperature Stability According to the Presence or Absence of Calcium-Alginate Beads

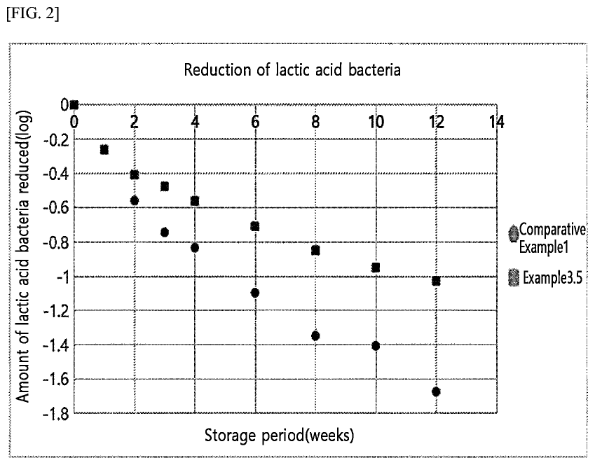

An attempt was made to compare the high-temperature stability of Example 6 and the Comparative Example. Specifically, the samples of the Comparative Example and Example 6, which were freeze-dried and powdered, were individually packaged and sealed at a fixed amount in aluminum pouch packaging. Each sample was stored in an incubator at 40° C. for 12 weeks. The reduction in lactic acid bacteria for each storage period is shown in FIG. 2.

As a result, as shown in FIG. 2, the amount of lactic acid bacteria was decreased by 1.674 log in the Comparative Example, while the amount of lactic acid bacteria was decreased by 1.027 log in Example 6, confirming that the high-temperature stability of the lactic acid bacteria prepared by the method of the present disclosure was improved.

### Experimental Example 4. Evaluation of Stability in the Gastrointestinal Tract According to the Presence or Absence of Calcium-Alginate Beads

An attempt was made to compare the stability in the gastrointestinal tract of Example 6 and the Comparative Example.

Specifically, the survival rates were compared through acid resistance and bile resistance tests according to simulated stomach duodenum passage (SSDP).

For the acid resistance test, 5N hydrochloric acid (HCl) was added to MRS broth to prepare acidic MRS broth at pH 3. For the bile resistance test, a 10% Oxgall solution and an artificial bile solution buffer were prepared.

In order to compare the survival rate of lactic acid bacteria, the freeze-dried powder prepared in the Comparative Example and the freeze-dried powder prepared in Example 6 were added to the acidic MRS and reacted at 37° C. for 1 hour. Thereafter, the 10% Oxgall solution and the buffer were added thereto to adjust the pH to 7, reacted for 2 hours, and then sampled to confirm the survival rate of lactic acid bacteria. The results are shown in Table 15 below.

TABLE 15

| | Initial Number | | |
|---|---|---|---|
| | of Bacteria | Test Results | Survival Rate |
| | (cfu/g) | (cfu/g) | (%) |
| Comparative Example | 2.45.E+11 | 9.02.E+08 | 0.37 |
| Example 6 | 9.16.E+10 | 3.21.E+10 | 34.94 |

With reference to Table 15 above, the lactic acid bacteria of the Comparative Example showed a survival rate of 0.37%, while the lactic acid bacteria encapsulated with calcium-alginate beads of Example 6 showed a survival rate of 34.94%, confirming that the acid resistance and bile resistance were greatly improved.

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

1. A method for producing lactic acid bacteria encapsulated with calcium-alginate beads, comprising:
(a) culturing lactic acid bacteria and recovering bacterial cells;
(b) mixing the lactic acid bacteria of step (a) with an alginate-containing solution; and
(c) adding the mixture of step (b) to a calcium-containing solution to form calcium-alginate beads,
wherein the alginate-containing solution comprises sucrose, sorbitol, or soy peptone, and the calcium-containing solution comprises trehalose or maltodextrin.

2. The method of claim 1, further comprising (d) freeze-drying the lactic acid bacteria encapsulated with the calcium-alginate beads of step (c).

3. The method of claim 1, wherein the lactic acid bacteria comprise at least one selected from the group consisting of Lactobacillus sp., Bifidobacterium sp., Streptococcus sp., Lactococcus sp., Enterococcus sp., Pediococcus sp., Leuconostoc sp., and Weissella sp.

4. The method of claim 1, wherein the lactic acid bacteria comprise at least one selected from the group consisting of Lactobacillus plantarum, Lactobacillus casei, Lactobacillus rhamnosus, Lactobacillus acidophilus, Bifidobacterium bifidum, Bifidobacterium longum, Bifidobacterium breve, Streptococcus faecalis, and Lactococcus lactis subsp. lactis.

5. The method of claim 1, wherein the lactic acid bacteria comprise at least one selected from the group consisting of Lactobacillus plantarum CJLP133, Lactobacillus plantarum CJLP243, Lactobacillus plantarum CJLP136, Lactobacillus plantarum CJLP55, and Lactobacillus plantarum CJLP56.

6. A lactic acid bacteria encapsulated with calcium-alginate beads produced by the method of any one of claim 1.

7. The lactic acid bacteria of claim 6, wherein the lactic acid bacteria have improved survival rate after freeze-drying compared to that of lactic acid bacteria which are not encapsulated with calcium-alginate beads.

8. The lactic acid bacteria of claim 6, wherein the lactic acid bacteria have improved stability compared to that of a lactic acid bacteria which are not encapsulated with calcium-alginate beads.

9. A method for improving the production yield of calcium-alginate beads for encapsulating lactic acid bacteria, comprising:
(a) culturing lactic acid bacteria and recovering bacterial cells;
(b) mixing the lactic acid bacteria of step (a) with an alginate-containing solution; and
(c) adding the mixture of step (b) to a calcium-containing solution to form calcium-alginate beads,
wherein the alginate-containing solution comprises sucrose, sorbitol, or soy peptone, and the calcium-containing solution comprises trehalose or maltodextrin.

10. A method of improving the survival rate of freeze-dried lactic acid bacteria, comprising:
(a) culturing lactic acid bacteria and recovering bacterial cells;
(b) mixing the lactic acid bacteria of step (a) with an alginate-containing solution;
(c) adding the mixture of step (b) to a calcium-containing solution to form calcium-alginate beads; and
(d) freeze-drying the lactic acid bacteria encapsulated with the calcium-alginate beads of step (c),
wherein the alginate-containing solution comprises sucrose, sorbitol, or soy peptone, and the calcium-containing solution comprises trehalose or maltodextrin.
